(19) United States
(12) Patent Application Publication
(10) Pub. No.: US 2011/0085968 A1
(43) Pub. Date: Apr. 14, 2011

(54) ARTICLES COMPRISING NANO-MATERIALS FOR GEOMETRY-GUIDED STEM CELL DIFFERENTIATION AND ENHANCED BONE GROWTH

(75) Inventors: Sungho JIN, San Diego, CA (US); Seunghan OH, Jeon Ju (KR); Karla BRAMMER, San Diego, CA (US)

(73) Assignee: THE REGENTS OF THE UNIVERSITY OF CALIFORNIA, Oakland, CA (US)

(21) Appl. No.: 12/900,249

(22) Filed: Oct. 7, 2010

Related U.S. Application Data
(60) Provisional application No. 61/251,135, filed on Oct. 13, 2009.

(51) Int. Cl. A61F 2/00; A61K 35/12; A61K 39/395; A61K 51/00; A61K 49/00; A61P 19/00; B82Y 5/00

(52) U.S. Cl. 424/1.11; 424/423; 424/93.7; 424/130.1; 424/9.1; 977/742; 977/762

(57) ABSTRACT

The present invention provides articles of manufacture comprising biocompatible nanostructures comprising significantly increased surface area for, e.g., organ, tissue and/or cell growth, e.g., for bone, tooth, kidney or liver growth, and uses thereof, e.g., for in vitro testing of drugs, chemicals or toxins, or as in vivo implants, including their use in making and using artificial tissues and organs, and related, diagnostic, screening, research and development and therapeutic uses, e.g., as drug delivery devices. The present invention provides biocompatible nanostructures with significantly increased surface area, such as with nanotube and nanopore array on the surface of metallic, ceramic, or polymer materials for enhanced cell and bone growth, for in vitro and in vivo testing, cleansing reaction, implants and therapeutics. The present invention provides optically transparent or translucent cell-culturing substrates. The present invention provides biocompatible and cell-growth-enhancing culture substrates comprising elastically compliant protruding nanostructure substrates coated with Ti, TiO₂ or related metal and metal oxide films.

### CROSS REFERENCE TO RELATED APPLICATIONS

This U.S. utility patent application claims benefit of priority to U.S. Provisional Patent Application Ser. No. 61/251,135, filed Oct. 13, 2009. The contents of this application is expressly incorporated herein by reference in its entirely for all purposes.

### FIELD OF THE INVENTION

The present invention provides articles of manufacture comprising biocompatible nanostructures which in alternative embodiments are used to guide or induce the differentiation of stem cells, or guide and induce the activity of osteoblasts and deposition of bone; and methods for making and using them. In alternative embodiments, compositions of the invention comprise nanostructure substrates coated with Ti, TiO₂ and/or related metal and metal oxide films.

### BACKGROUND OF THE INVENTION

Ti and Ti alloys are corrosion resistant, light, yet sufficiently strong for load-bearing, and are machinable. They are one of the few biocompatible metals which osseo-integrate (direct chemical or physical bonding with adjacent bone surface without forming a fibrous tissue interface layer). For these reasons, they have been used successfully as orthopedic and dental implants.

Patients who go through Ti implant operations for repair of hip joints, broken bones, or dental implants often have to wait for many months of slow bone growth recovery before they are cured enough to get off the confinement on a bed or crutches and have a normal life. Accelerated bone growth would thus be very beneficial for such patients.

One of the important goals in stem cell research for orthopedic applications is to control and direct the differentiation of stem cells, in particular, human mesenchymal stem cells (hMSCs) into a specific cell lineage, desirably osteoblast cells.

Many biochemists and molecular biologists induce differentiation by chemical factors in vitro; however there is no well-understood way of controlling such factors and precise concentrations in vivo. In fact, the side effects of many chemicals such as dexamethasone or β-glycerophosphate, typical osteogenic inducing reagents, are not known because the body does not encounter these types of chemicals naturally. The dexamethasone is actually one of the well known component of toxic cancer chemotherapy drug.

### SUMMARY

In alternative embodiments the invention provides products of manufacture comprising a biocompatible surface, wherein at least a portion of (or at least part of) (e.g., at least about 0.5%, 1%, 2%, 3%, 4%, 5%, 6%, 7%, 8%, 9% or 10% or more), or all of (or the entirety, or 100% of), the surface area of the biocompatible surface comprises or is covered or coated by a structure or structures comprising:
(a) (i) a plurality of nanotubular structures that are between about 70 to 200 nanometers (nm) in diameter, or between about 60 to 150 nm in diameter,
   (ii) a plurality of nanowires, nano-lines or nano-grooves having a spacing of between about 70 to 200 nanometers (nm), or between about 60 to 150 nm, or
   (iii) a combination (in any proportion or combination) of the nanotubular structures of (i) and the nanowires, nano-lines or nano-grooves of (ii);
(b) the product of manufacture of (a), wherein a portion of, or all of, the nanotubular structures comprise nanotubes;
(c) the product of manufacture of (a) or (b), wherein the nanotubular structures, nanowires, nano-lines or nano-grooves comprise a metal or a metal alloy, and/or a stainless steel or a ceramic, and/or the biocompatible surface (e.g., as a coating) comprises a metal or a metal alloy or metal oxide (e.g., as a single or alloyed oxide), and/or a stainless steel or a ceramic, and/or a polymer;
(d) the product of manufacture of (c), wherein the metal or a metal alloy comprises: Ti, Zr, Hf, Nb, Ta, Mo and/or W metal material; a Ti, Zr, Hf, Nb, Ta, Mo and/or W alloy; a Ti, Zr, Hf, Nb, Ta, Mo and/or W single or alloyed oxide,
wherein the metal, metal alloy, metal material, single or alloyed oxide comprise at least part of (e.g., at least about 1%, 2%, 3%, 4%, 5%, 6%, 7%, 8%, 9% or 10% or more) or the entire (e.g., as completely, or 100%) materials content of a nanostructure, or are manufactured as all (e.g., as completely, or 100%) or part of (e.g., at least about 1%, 2%, 3%, 4%, 5%, 6%, 7%, 8%, 9% or 10% or more) a surface coating with between about 2 to 20 nm, or between about 1 to 40 nm, or between about 0.5 to 60 nm, thickness on the nanostructures (e.g., nanotubes, nanowires, nano-lines or nano-grooves);
(e) the product of manufacture of any of (a) to (d), wherein the nanotubular structures, nanowires, nano-lines or nano-grooves are straight, curved and/or bent, and optionally the nanotubular structures, nanowires, nano-lines or nano-grooves are fixed or loosely placed, or a combination thereof, on the biocompatible surface (which can be a surface coating);
(f) the product of manufacture of any of (a) to (e), wherein at least a portion of (least part of) (e.g., at least about 1%, 2%, 3%, 4%, 5%, 6%, 7%, 8%, 9% or 10% or more), or all of (e.g., 100% of), the nanotubular structures, nanowires, nano-lines and/or nano-grooves are arranged as an array, and optionally the nanotubular structures, nanowires, nano-lines and/or nano-grooves are arranged as three-dimensional network scaffolds;
(g) the product of manufacture of any of (a) to (f), wherein at least a portion of (least part of) (e.g., at least about 1%, 2%, 3%, 4%, 5%, 6%, 7%, 8%, 9% or 10% or more), or all of (e.g., 100% of), the nanotubular structures, nanowires, nano-lines or nano-grooves are in contact with or are contained within or upon (e.g., in contact with the surface of) or within the immediate vicinity of a cell, wherein optionally the cell is suitable for implantation and/or regeneration of a bone and/or a joint tissue in a subject, and optionally the cell is an osteoblast, a stem cell or a mesenchymal stem cell (MSC);
(h) the product of manufacture of any of (a) to (g), wherein at least a portion of (least part of) (e.g., at least about 1%, 2%, 3%, 4%, 5%, 6%, 7%, 8%, 9% or 10% or more), or all of (e.g., 100% of), the nanotubular structures, nanowires, nano-lines or nano-grooves comprise or further comprise (or are associated with or covalently or non-covalently bound to) one or more active agents, biologically active agents, or an osteogenic inducing agent, or a therapeutic drug, a growth factor, a protein, an enzyme, a hormone, a nucleic acid, an RNA, a DNA, a gene, a vector, a phage, an antibiotic, an antibody, a small molecule, a radioisotope, a magnetic nanoparticle and/or a particle;
(i) the product of manufacture of any of (a) to (h), wherein the nanotubular structures, nanowires, nano-lines or nano-grooves are between about 70 to 200 nanometers (nm) in diameter or width, or between about 60 to 150 nm, or between about 40 to 160 nm, in diameter or width, or have a spacing between the nanowires, nano-lines or nano-grooves of between about 70 to 200 nanometers (nm), or between about 60 to 150 nm; and optionally the nanotubular structures, nanowires, nano-lines or nano-grooves are in contact with or are contained within or upon (e.g., in contact with the surface of) or within the immediate vicinity of a cell, a stem cell, a mesenchymal stem cell (MSC) or a human mesenchymal stem cell (hMSC); or embryonic stem cell, or
(j) the product of manufacture of any of (a) to (h), wherein the nanotubular structures, nanowires, nano-lines or nano-grooves are about 90, 95, 100, 105 or 110 or more nanometers (nm) in diameter or width, or are between about 80 to 120 nm, or between about 60 to 140 nm, in diameter or width, or have a spacing between the nanowires, nano-lines or nano-grooves of about 100 nanometers (nm), or have a spacing between them of about 80 to 120 nm, or between about 60 to 140 nm; and optionally the nanotubular structures, nanowires, nano-lines or nano-grooves are in contact with or are contained within or upon (e.g., in contact with the surface of) or within the immediate vicinity of a cell, or a stem cell, or a mesenchymal stem cell (MSC) or a human mesenchymal stem cell (hMSC), or an embryonic stem cell, or a “regular” or fully differentiated or partially differentiated osteoblast cell.

In alternative embodiments at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or all, of the biocompatible surface (which can be a surface coating) is covered by a plurality of nanotubular structures, nanowires, nano-lines or nano-grooves. In alternative embodiments the biocompatible surface (which can be a surface coating) and/or the nanotubular structures, nanowires, nano-lines or nano-grooves can comprise: (i) a matrix material; or a Ti, Zr, Hf, Nb, Ta, Mo and/or W metal; or (ii) an alloy or a single or alloyed oxide of a Ti, Zr, Hf, Nb, Ta, Mo and/or W metal; (iii) an alloy of a Ti, Zr, Hf, Nb, Ta, Mo and/or W metal; or a Si, a Si oxide, an Al, an Al oxide, a carbon, a diamond, a noble metal, an Au, an Ag, a Pt and/or an Al, Au, an Ag, a Pt alloy, a polymer or a plastic material, a composite metal, a ceramic, a polymer and/or a combination thereof.

In alternative embodiments the biocompatible surface has a thin film coating of between about 2 to 20 nm, or between about 1 to 40 nm, or between about 0.5 to 60 nm thickness, wherein in alternative embodiments the coating comprises in part (e.g., at least about 0.5%, 1%, 2%, 3%, 4%, 5%, 6%, 7%, 8%, 9% or 10% or more) or in its entirety (e.g., 100%) Ti, Zr, Hf, Nb, Ta, Mo and/or W metal; or an oxide (or a single or alloyed oxide) of a Ti, Zr, Hf, Nb, Ta, Mo and/or W metal; or an alloy or an alloyed oxide of a Ti, Zr, Hf, Nb, Ta, Mo and/or W metal.

In alternative embodiments the product of manufacture further comprises a bone cell, a liver cell, a kidney cell, a blood vessel cell, a skin cells, a periodontal cell or a periodontal tissue cell, a stem cell, an organ cell, or wherein the cell is a bone cell, a liver cell, a kidney cell, a blood vessel cell, a skin cells, an organ cell. In alternative embodiments the product of manufacture further comprises a plurality of cells, wherein the cells comprise bone cells, liver cells, liver parenchymal cells, endothelial cells, adipocytes, fibroblastic cells, Kupffer cells, kidney cells, blood vessel cells, skin cells, periodontal cells, odontoblasts, dentinoblasts, cementoblasts, enameloblasts, odontogenic ectomesenchymal tissue, osteoblasts, osteoclasts, fibroblasts, and other cells and tissues involved in odontogenesis or bone formation and/or stem cells, and other human or animal organ cells, or the cells are embryonic or adult stem cells, or a combination thereof.

In alternative embodiments the cell is a human or an animal cell, or the product of manufacture, further comprises a human or an animal cell.

In alternative embodiments the products of manufacture further comprise a hydroxyapatite, a bio-degradable polymer, or a bio-compatible or bio-inert bone cement; or further comprising a biological agent or a therapeutic composition, or an osteogenic inducing agent, or a growth factor, a collagen, a nucleic acid, an antibiotic, a hormone, a drug, a magnetic particle, a metallic particle, a ceramic particle, a polymer particle and/or a drug delivery particle.

In alternative embodiments the nanotubular structures or nanotubes are made by anodization or by patterned chemical etching or a combination thereof. In alternative embodiments the products of manufacture further comprise a nanodepot or microdepot, either the nanotube inside holes themselves (e.g., between about 70 to 200 nm diameter, or between about 50 to 300 nm diameter) or separately patterned and distributed micrometer-scale pores or grooves (e.g., between about 1 to 1,000 micrometer diameter).

In alternative embodiments the nanotubular structures or nanotubes comprise between about 1 to 1,000 micrometer diameter nanostructures (e.g., nanotubes) to be able to contain therein a cell, e.g., a stem cell, e.g., a 5 to 50 micrometer size stem cell. Thus, in addition to the general nanotube structures of the invention, alternative embodiment comprise products of manufacture, e.g., nanostructures, e.g., nanotubes, having between about 1 to 1,000 micrometer size cavities/holes/indentations (and the like) on the implant surface (which can cover only a portion or part of (e.g., at least about 1%, 2%, 3%, 4%, 5%, 6%, 7%, 8%, 9% or 10% or more), or all of (e.g., 100% of) the cell, e.g., the stem cell. Thus, alternative embodiments can house or “store” a cell, e.g., a stem cell, so that they can be provided or released whenever needed. In alternative embodiments this provides additional amounts of cells, e.g., stem cells, where needed, as compared to a naturally very small number of body-supplied stem cells near the implant]comprising a metallic or oxide material, hydroxyapatite, a bio-degradable polymer, or a bio-compatible or bio-inert bone cement; or further comprising, or an osteogenic inducing agent, or a biological agent or a therapeutic composition, or a growth factor, a collagen, a nucleic acid, an antibiotic, a hormone, a drug, a magnetic particle, a metallic particle, a ceramic particle, a polymer particle and/or a drug delivery particle.

In alternative embodiments the matrix material providing the nanodepot or microdepot, either the nanotube inside holes themselves (e.g., 70-200 nm diameter) or separately patterned and distributed micrometer-scale pores or grooves (e.g., 1-1,000 micrometer diameter), is/are made of (comprise) metal, oxide, hydroxyapatite, bio-degradable polymer, bio-compatible or bio-inert bone cement. In alternative embodiments, one or more of components comprise a cell, a stem cell, an osteogenic inducing agent, an agent or a biological agent or a therapeutic composition, or a growth factor, a collagen, a nucleic acid, an antibiotic, a hormone, a drug, a magnetic particle, a metallic particle, a ceramic particle, a polymer particle or a drug delivery particle, or any combination thereof, are stored in (e.g., contained in) a nanodepot or microdepot, e.g., a nanotubular structure or a nanotube cavity, or are stored in (e.g., contained in) a spacing between adjacent nanostructures, e.g., a nanotubular structure, a nanowire, a nano-line and/or a nano-groove, or are stored in (e.g., contained in) a microscale pore or groove (e.g., 1-1,000 micrometer diameter), which in alternative embodiments are distributed on a surface of a product of manufacture of the invention, e.g., an implant surface, depending on the size or structure of the agent, cell and/or composition, e.g., a biological agent or a stem cell, to be stored or contained therein.

In alternative embodiments the invention provides composition delivery devices comprising a product of manufacture of the invention, wherein optionally the composition comprising a hydroxyapatite, a bio-degradable polymer, or a bio-compatible or bio-inert bone cement; or further comprising, or stem cells, an osteogenic inducing agent, or a biological agent or a therapeutic composition, or a growth factor, a collagen, a nucleic acid, an antibiotic, a hormone, a drug, a magnetic particle, a metallic particle, a ceramic particle, a polymer particle and/or a drug delivery particle.

In alternative embodiments the invention provides medical implants, orthopedic (orthopedic) implants or dental implants comprising a product of manufacture of the invention, and optionally the orthopedic (orthopedic) implant or dental implant comprises a plurality of cells, and optionally the cells comprise bone cells, liver cells, liver parenchymal cells, endothelial cells, adipocytes, fibroblastic cells, Kupffer cells, kidney cells, blood vessel cells, skin cells, periodontal cells, odontoblasts, dentinoblasts, cementoblasts, enameloblasts, odontogenic ectomesenchymal tissue, osteoblasts, osteoclasts, fibroblasts, and other cells and tissues involved in odontogenesis or bone formation and/or stem cells, and other human or animal organ cells, or the cells are embryonic or adult stem cells, or a combination thereof.

In alternative embodiments the invention provides artificial tissues or organs comprising a product of manufacture of the invention, and optionally the artificial tissue or organ comprises a plurality of cells, and optionally the cells comprise bone cells, liver cells, liver parenchymal cells, endothelial cells, adipocytes, fibroblastic cells, Kupffer cells, kidney cells, blood vessel cells, skin cells, periodontal cells, odontoblasts, dentinoblasts, cementoblasts, enameloblasts, odontogenic ectomesenchymal tissue, osteoblasts, osteoclasts, fibroblasts, and other cells and tissues involved in odontogenesis or bone formation and/or stem cells, and other human or animal organ cells, or the cells are embryonic or adult stem cells, or a combination thereof.

In alternative embodiments the invention provides methods for guiding, controlling and/or directing the differentiation of a stem cell, or accelerating the differentiation and directing the differentiation of a stem cell, comprising implanting, growing and/or culturing the stem cell in or on (or on the surface of) a product of manufacture of the invention. The methods method can be for guiding, controlling and/or directing the differentiation of the stem cell, or accelerating and directing the differentiation of a mesenchymal stem cell (MSC) or a human mesenchymal stem cell (hMSC).

In alternative embodiments of the methods the nanotubular structures, nanowires, nano-lines or nano-grooves are between about 70 to 200 nanometers (nm) in diameter or width, or between about 60 to 150 nm in diameter or width, or have a spacing between the nanowires, nano-lines or nano-grooves of between about 70 to 200 nanometers (nm), or between about 60 to 150 nm.

In alternative embodiments of methods of the invention cells, e.g., stem cells, e.g., mesenchymal stem cell (MSC) or the human mesenchymal stem cell (hMSC), are provided to (e.g., provided approximate to or near or adjacent to) an implant location e.g., by injection, by mixing with a composite liquid comprising viscosity-enhancing medium such as a bioinert polymer or a biodegradable polymer in liquid or particle form, by adding to a bone cement, by inserting/storing into nanopores in the nanotubes, and/or by inserting/storing in microcavities or microdepots prepared on the surface of a product of manufacture of the invention, e.g., on an implant surface. In alternative embodiments these cells, e.g., stem cells, e.g., mesenchymal stem cells are directed to differentiate into an osteoblast, or a cell of an osteogenic lineage.

In alternative embodiments of the methods the product of manufacture is placed or implanted in vivo, and the mesenchymal stem cell (MSC) or the human mesenchymal stem cell (hMSC) differentiates either before in vivo implantation, after in vivo implantation, or a combination thereof.

In alternative embodiments the invention provides methods for inducing, enhancing and/or prolonging the bone-forming capacity of a “regular”, or a fully differentiated or a partially differentiated osteoblast cell or cell of osteogenic lineage, comprising implanting, growing and/or culturing an osteoblast cell or a cell of osteogenic lineage in a product of manufacture of the invention. The nanotubular structures, nanowires, nano-lines or nano-grooves can be about 100 nanometers (nm) in diameter or width, or are between about 80 to 120 nm in diameter or width, or have a spacing between the nanowires, nano-lines or nano-grooves of about 100 nanometers (nm), or have a spacing between them of about 80 to 120 nm.

In alternative embodiments of the methods the product of manufacture is placed or implanted in vivo.

In alternative embodiments of the methods the metallic or oxide (e.g., single or alloyed oxide) matrix, hydroxyapatite, bio-degradable polymer, bio-compatible or bio-inert bone cement contains nanostructures, or biological agents or therapeutic compositions, or growth factors, collagen, nucleic acid, antibiotic, hormone, drugs, magnetic particles, metallic particles, ceramic particles, polymer particle, drug delivery particles and the like, and these compositions can be stored in the nanotubular structures or the nanotube cavities, or stored in spacing between adjacent nanowires nanotubular structures, nanowires, nano-lines or nano-grooves, or released by a remote-controlled mechanism or device, and optionally the remote-controlled mechanism or device comprises use of an electric field, a magnetic field, an AC magnetic field, ultrasonic agitation, or infrared light stimulation.

In alternative embodiments of the methods the guiding, controlling and/or directing of stem cell differentiation and/or inducing, enhancing and/or prolonging of bone formation is improved, accelerated or prolonged by combining with a full-dose or partial use of an osteogenic inducing agent.

In alternative embodiments the invention provides methods for selectively releasing a therapeutic, an imaging, a drug or a biological agent in a subject, the method comprising
(a) implanting a product of manufacture of the invention, in a subject, wherein the product of manufacture comprises a therapeutic, an imaging, a drug or a biological agent in a liquid or colloidal composition; and,
(b) contacting the product of manufacture with ultrasonic or magnetic agitation of the liquid or colloidal composition, wherein the biological agent is released from the product of manufacture;
and optionally the magnetic nanoparticle is selected from the group consisting of iron-oxide particles of magnetite (Fe₃O₄) or maghemite (γ-Fe2O3), and optionally the magnetic nanoparticle is about 5 to 50 nm in average diameter.

In alternative aspects, the compositions of the invention are used as bio- or in vivo implants with protruding structural features above the implant surface; and in alternative embodiments these can provide advantageous characteristics for bio applications such as further improved stability, biocompatibility and mechanical lock-in reliability at the implant-cultured bone/cell interface, as well as substantially accelerated cell/bone growth accelerating characteristics due to the TiO₂ nanotube and related structures.

In alternative aspects, compositions of the invention have protruding nanostructures in a configuration of a periodic or a random array of nanopillars, nanoballs, nanolines or nanomesh elements, or a combination thereof.

In alternative embodiments, the invention provides kits comprising compositions and methods of the invention, including products of manufacture of the invention comprising cells, biological agents, transfecting agents, transducing agents, instructions (regarding the methods of the invention), or any combination thereof. As such, kits, cells (e.g., a mammalian cell, e.g., a stem cell, e.g., a mesenchymal stem cell (MSC) such as a human mesenchymal stem cell), vectors and the like are provided herein.

The details of one or more embodiments of the invention are set forth in the accompa-nying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### BRIEF DESCRIPTION OF THE DRAWINGS

The drawings are illustrative of embodiments of the invention and are not meant to limit the scope of the invention as encompassed by the claims. The advantages, nature and additional features of the invention will appear more fully upon consideration of the illustrative embodiments described in the accompanying drawings.

FIG. 1 (a)-(d) illustrate SEM micrographs (as a top view) of exemplary self-aligned TiO₂ nanotubes of the invention with significantly different diameters (as indicated in the diagram, or, FIG. 1(a) is 30 nm, FIG. 1(b) is 50 nm, FIG. 1(c) is 70 nm, and FIG. 1(d) is 100 nm); FIG. 1 (e) illustrates an SEM micrograph (as an oblique view) of the exemplary 100 nm diameter TiO₂ nanotube of the invention; and FIG. 1(f) illustrates a cross-sectional transmission electron microscopy (TEM) of the exemplary 100 nm diameter TiO₂ nanotubes of the invention, as described in detail, below.

FIG. 2 illustrates SEM micrographs of human mesenchymal stem cells (hMSCs) on exemplary flat Ti and 30, 50, 70, 100 nm diameter (as labeled on the SEM micrograph panels) TiO₂ nanotube surfaces of the invention after 2 hours and 24 hours of culture, as described in detail, below.

FIG. 3 illustrates SEM micrographs showing extracellular matrix aggregates on the surfaces of exemplary flat Ti (top left SEM panel) and 30 nm (top middle SEM panel), 50 nm (top right SEM panel), 70 nm (bottom left SEM panel), 100 nm (bottom right SEM panel) diameter TiO₂ nanotubes of the invention after 2 hours of hMSCs culture, as described in detail, below.

FIG. 4 illustrates immunofluorescent images of osteopontin (OPN) (top middle panel) and osteocalcin (OCN) (bottom middle panel), as well as actin (upper and lower left panels) and DAPI (upper and lower right panels), on exemplary 100 nm diameter TiO₂ nanotubes of the invention after 3 weeks of culture, as described in detail, below.

FIG. 5 graphically illustrates data from a quantitative PCR analysis for alkaline phosphatase (ALP), osteocalcin (OCN), and osteopontin (OPN), as described in detail, below.

FIG. 6 is a schematic illustration of the overall trends of nano cue effects of products of manufacture of the invention on hMSC fate and morphology after 24 hr culture, as described in detail, below.

FIG. 7 graphically illustrates alkaline phosphatase activity of primary bone cells (osteoblast cells) cultured on flat Ti, and various sized TiO₂ nanotubes of the invention (at 30, 50, 70 and 100 nm), at 24 hour (h) (left) and 48 h (right) of incubation, as described in detail, below.

It is to be understood that the drawings are for purposes of illustrating the concepts of the invention and are not to scale.

### DETAILED DESCRIPTION

In alternative embodiments, the invention provides products of manufacture comprising biocompatible nanotubular structures that are between about 70 to 200 nanometers (nm) in diameter, or between about 60 to 150 nm in diameter. In alternative aspects, the nanotubular structures comprise nanotubes, and/or comprise Ti or Ti oxide nanotubular structures, such as Ti or Ti oxide nanotubes. In alternative aspects, these products of manufacture further comprise a cell such as a mammalian or a human cell, e.g., a stem cell, e.g., a mesenchymal stem cells (hMSCs), e.g., human mesenchymal stem cells (hMSCs).

In alternative embodiments, the invention provides methods for using products of manufacture of the invention comprising biocompatible nanostructures (e.g., nanotubular structures, and/or nanowires, nano-lines or nano-grooves) using a cell such as a mammalian or a human cell, e.g., using stem cells, for example, in cell implant, tissue regeneration and/or stem cell therapies or research, e.g., for tissue regeneration or restoration or orthopedic applications, e.g., for inducing cartilage and/or bone growth.

In alternative embodiments, these compositions of the invention are used to control and direct the differentiation of a cell, e.g., a mammalian cell, e.g., a stem cell, e.g., a mesenchymal stem cell (MSC) such as human mesenchymal stem cells (hMSCs), into a specific cell lineage, such as osteoblast cells or cells for cartilage growth.

In alternative embodiments, compositions of the invention have (comprise) nanotubular structures that are at least about 70 to 200 nanometers (nm) in diameter, or between about 60 to 150 nm in diameter, and can be used to control and direct the differentiation of a cell, e.g., a mammalian cell, e.g., a stem cell, e.g., a mesenchymal stem cell (MSC) such as human mesenchymal stem cells (hMSCs), into a specific cell lineage, such as osteoblast cells or cells for cartilage growth. In one aspect these nanotubular structures comprise Ti oxide nanotubes, or nanotubular structures comprising single or alloyed metal oxides.

In alternative embodiments, compositions of the invention having nanotubular structures that are at least about 100 nanometers (nm) in diameter, or between about 80 to 120 nm in diameter, are used to induce, enhance or prolong the bone-forming capacity of “regular” (e.g., already differentiated or non-stem cell induced) osteoblast cells. In one aspect, these nanotubular structures comprise Ti oxide nanotubes.

In alternative embodiments of methods of the invention cells, e.g., stem cells, e.g., mesenchymal stem cells (MSC) or human mesenchymal stem cells (hMSC), are provided to or near an implant location e.g., by injection, by mixing with a composite liquid comprising viscosity-enhancing medium such as a bioinert polymer or a biodegradable polymer in liquid or particle form, by adding to a bone cement, by inserting/storing into nanopores in the nanotubes, or by inserting/storing in microcavities prepared on the implant surface. These cells, e.g., stem cells, e.g., mesenchymal stem cells (MSC) or human mesenchymal stem cells (hMSC), are directed to differentiate into an osteoblast, or a cell of an osteogenic lineage.

The invention will be further described with reference to the following examples; however, it is to be understood that the invention is not limited to such examples.

### EXAMPLES

### Example 1

### Compositions of the Invention can Induce a Specific and Preferred Differentiation of Mesenchymal Stem Cells (MSCs)

We have demonstrated that the compositions of the invention are effective for inducing a specific and preferred differentiation of mesenchymal stem cells (MSCs), e.g., human MSCs (hMSCs), into osteoblasts using only the geometric cues of the nanotubular structures' diameter; and in one aspect this specific and preferred differentiation of MSCs (e.g., hMSCs) into osteoblasts (as induced by compositions of the invention) occurs absent osteogenic inducing environmental cues, e.g., osteogenic-inducing media. In one embodiment, the osteoblast-inducing compositions of the invention comprise nanotubular-shaped titanium oxide surface structures on Ti implants and in alternative embodiments, comprising a plurality of nanotubular structures that are between about 70 to 200 nanometers (nm) in diameter, or between about 60 to 150 nm in diameter

hMSC behavior in response to defined nanotube sizes revealed a very dramatic change in hMSC behavior in a relatively narrow range of nanotube dimensions. Small (approximately 30 nm diameter) nanotubes promoted adhesion without noticeable differentiation, while larger (approximately 70-100 nm diameter) nanotubes of the invention elicited a dramatic stem cell elongation (approximately ten-fold increase), which induced cytoskeletal stress and selective differentiation into osteoblast-like cells. In alternative embodiments, use of compositions of the invention can bring about enhanced bone formation from primary osteoblast bone cells (e.g., as assayed by increased alkaline phosphatase activity) by at least about 20% compared with smaller diameter nanotubes. Accordingly, these studies demonstrate that the compositions of the invention provide a nanotechnology-based route for novel MSC applications, e.g., tissue regeneration, e.g., orthopedics-related hMSC treatments and enhanced bone growth.

It is also demonstrated that the bone-forming capacity of “regular” (e.g., already differentiated, non-stem cell induced) osteoblast cells is also substantially and desirably improved by selecting the diameter of the Ti oxide nanotubes to be at least approximately 70 nm diameter. We demonstrated that TiO₂ nanotubes size can regulate human mesenchymal stem cell (hMSC) differentiation towards an osteoblast lineage, in the absence of osteogenic inducing factors.

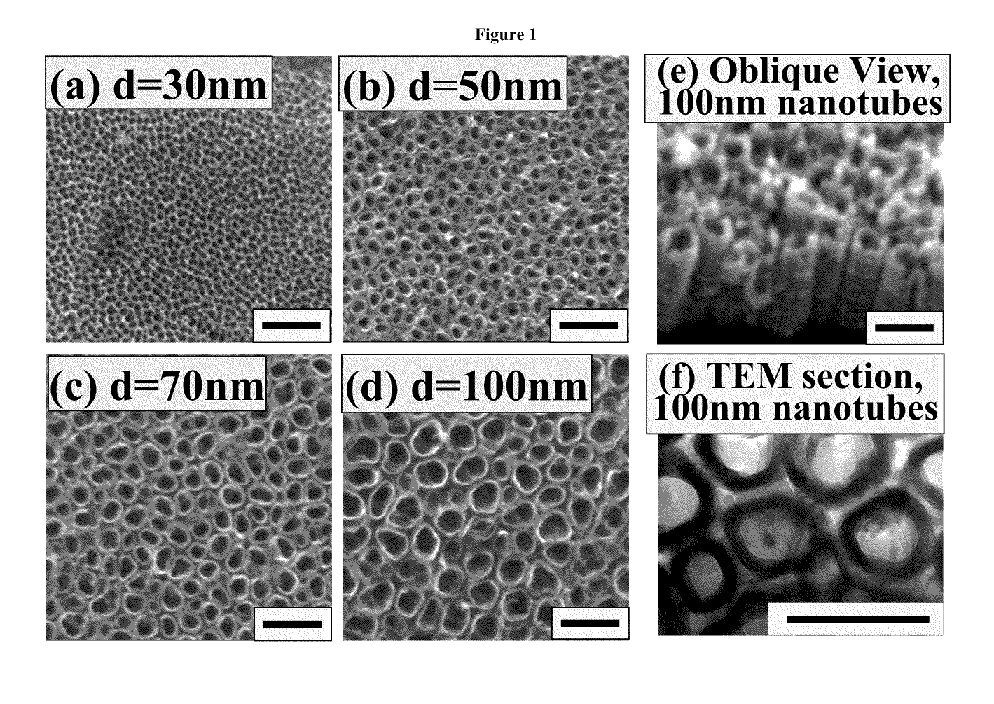

In order to determine how the size of the nanotubes would play a role in the cellular response and differentiation of hMSCs, 30, 50, 70, and 100 nm TiO₂ nanotubes were constructed on Ti substrates by anodization as depicted in FIG. 1 (see below for further discussion of FIG. 1). The self-assembled TiO₂ nanotube arrays on Ti substrates have a robust and discrete shape, confirmed by Scanning electron microscopy (SEM) images. The SEM images show highly ordered nanotubes with four different pore sizes between 30 to 100 nm. These nanotubes were created by controlling anodizing potentials: 5, 10, 15 and 20 V. Flat Ti substrates having a native TiO₂ oxidation layer on the surface with analogous chemical composition of the nanostructured TiO₂ substrates were used as comparison substrata.

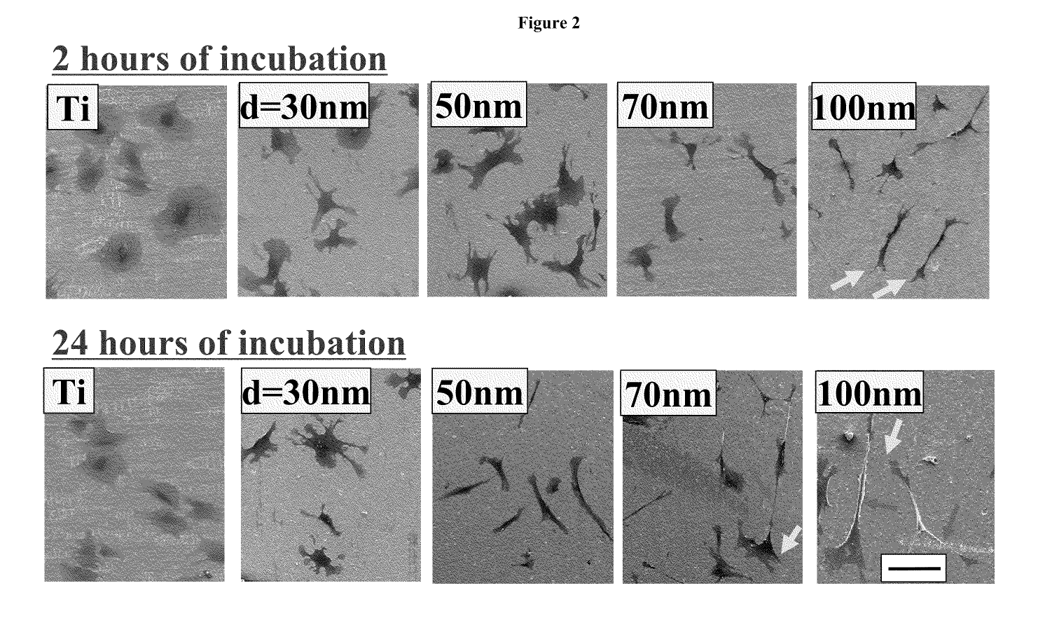

SEM images of hMSCs grown on these different diameter nanostructures showed that after 2 and 24 hrs of incubation the shape of the hMSCs sitting on top of flat Ti and on various sizes of TiO₂ nanotubes were all extremely different (see FIG. 2; see below for further discussion of FIG. 2). hMSCs on flat Ti appeared to be more round, stationary, and lacked the noticeable filopodia extensions and cellular propagation fronts like those cultures on TiO₂ nanotubes. In contrast, on nanotube surfaces particularly for the cases of larger diameter nanotubes of the invention, a large number of prominent filopodia and unidirectional lamellipodia extensions were apparent on the nanotube structures even after just 2 hrs of culture (FIG. 2, upper panels, see additional figure discussion, below). This trend becomes much more apparent if the hMSC culture time is extended to 24 hrs (FIG. 2, lower panels), and significantly more pronounced when the nanotube diameter is increased (lamellipodia are shown by the yellow arrows). As indicated by the red arrows, hMSCs exhibit cellular morphologies with extraordinary elongation of as much as 200 um in length on 100 nm TiO₂ nanotube surfaces. This is approximately a 10-fold increase in the hMSCs cellular elongation length as compared to the identical culture conditions for the 30 nm nanotubes.

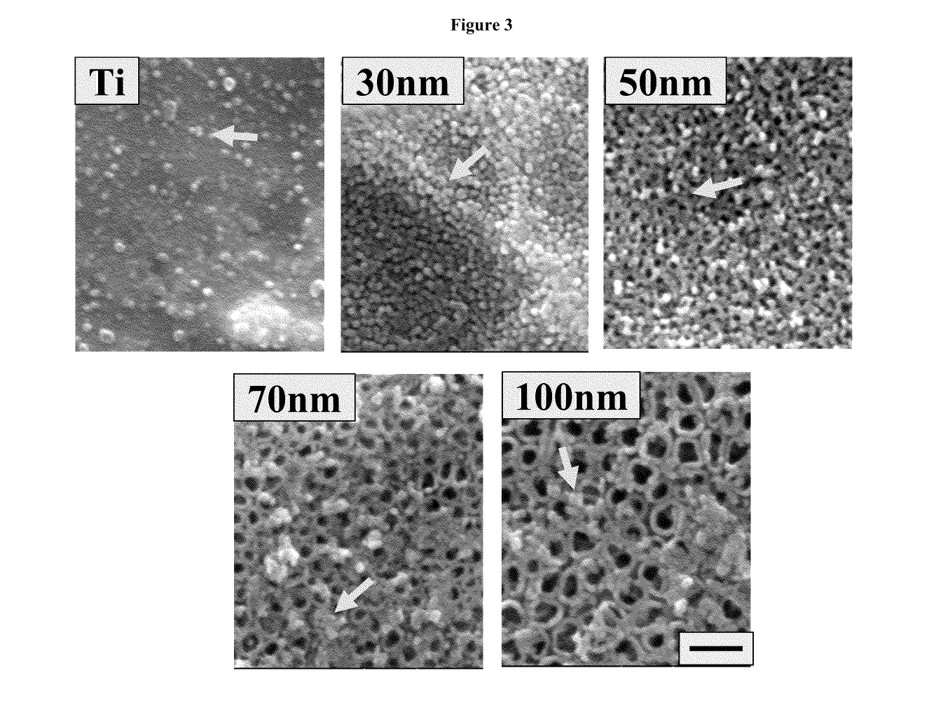

Higher magnification SEM images of TiO₂ nanotube substrata (FIG. 3, see additional figure discussion, below) after 2 hrs of culture reveal the adhesion of many round protein aggregates, approximately 30 nm in diameter, deposited from serum in the culture media. These protein aggregates are most likely settling on the surface, acting as a pre-existing, accessible protein coating, which would have implications on how the cells initially perceive the surface and the way they attach. Protein aggregates are rather quickly deposited after only 2 hrs of incubation on the wall top of the nanotubes, and immunofluorescent staining indicates that these aggregates are rich in fibronectin, an important extracellular matrix (ECM) protein that supports robust cell adhesion. Less protein aggregate deposition, with a sparse distribution, is observed on flat Ti samples, while aggregate coating of the Ti nanotubes directly scales with nanotube diameter.

The cell adhesion and elongation aspects were further studied to elucidate the nanotube size dependence of hMSC behavior. The number of adhered hMSCs on the experimental substrata decreased almost inversely proportional to the pore size of the TiO₂ nanotubes at the critical early stage of hMSC interaction with substrata (within the initial 24 hrs of incubation time). After prolonged incubation, cell growth for all other substrates caught up and these differences eventually were not as apparent due to the confluency of the cells, as might be anticipated at 7 days of culture time.

In order to understand the relationship between the nano dimensional cues and hMSC cell behavior, a quantitative analysis of cellular morphological elongation was also carried out. The elongation ratio of hMSCs increased with increasing size of TiO₂ nanotubes. The largest TiO₂ nanotubes showed an average elongation ratio of length/width as large as approximately 10, while the small diameter nanotubes of 30 nm (as well as the flat Ti) exhibited a basically isotropic configuration with an overall average elongation ratio of more or less approximately one (1). These cell elongation results were also confirmed with live cell imaging using FDA (fluorescein diacetate) staining. The extraordinary stretching of hMSCs for the larger diameter nanotubes of 70 and 100 nm of this invention demonstrate that cell elongation and adhesion appear to be inversely related to one another on Ti nanotubes.

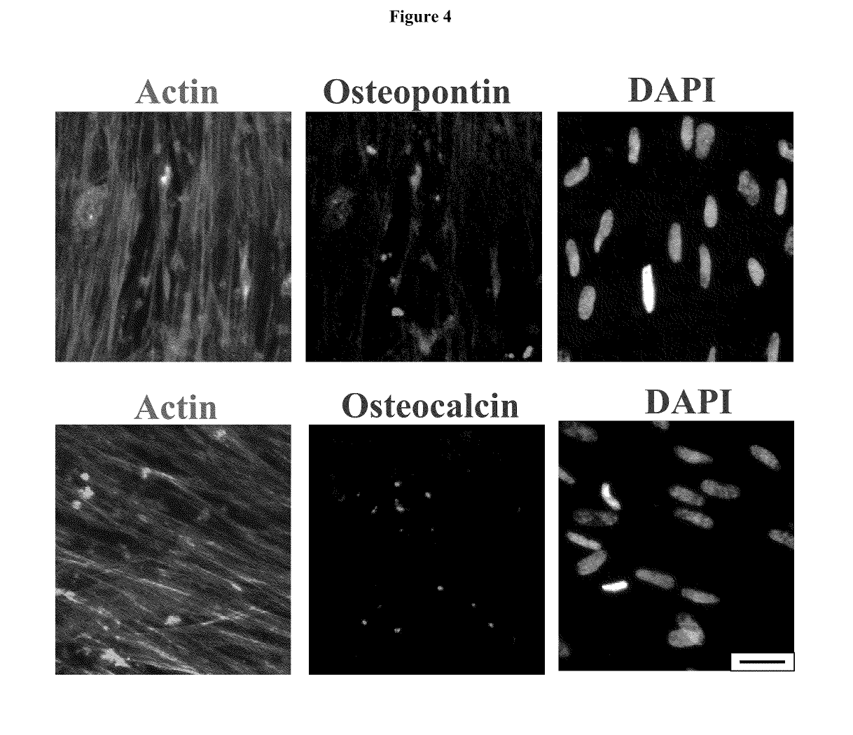

The elongation/stretching of the hMSCs on larger diameter nanotubes resulted in a preferential differentiation into osteogenic lineage, which was confirmed by immuno-fluorescent staining of two common protein osteogenetic markers: osteopontin (OPN) and osteocalcin (OCN). This analysis for the detection of osteogenic protein expression of hMSCs was conducted on cells cultured for 21 days. The immunofluorescent results, which are given in FIG. 4 (see additional figure discussion, below) for exemplary 100 nm TiO₂ nanotubes of the invention, showed that hMSCs had recognizable OCN and OPN positive staining with staining shapes associated with osteoblast cells, while hMSCs on the much smaller diameter nanotubes and flat Ti substrata did not elicit any positive staining. The upper panels of FIG. 4, represent osteopontin (OPN) staining (together with actin and DAPI, or 4′,6-diamidino-2-phenylindole, staining for the same image area to also illustrate the cytoplasmic actin skeleton and cell nuclei), while the lower panels show the osteocalcin (OCN) staining (again with actin and DAPI staining of the same image area).

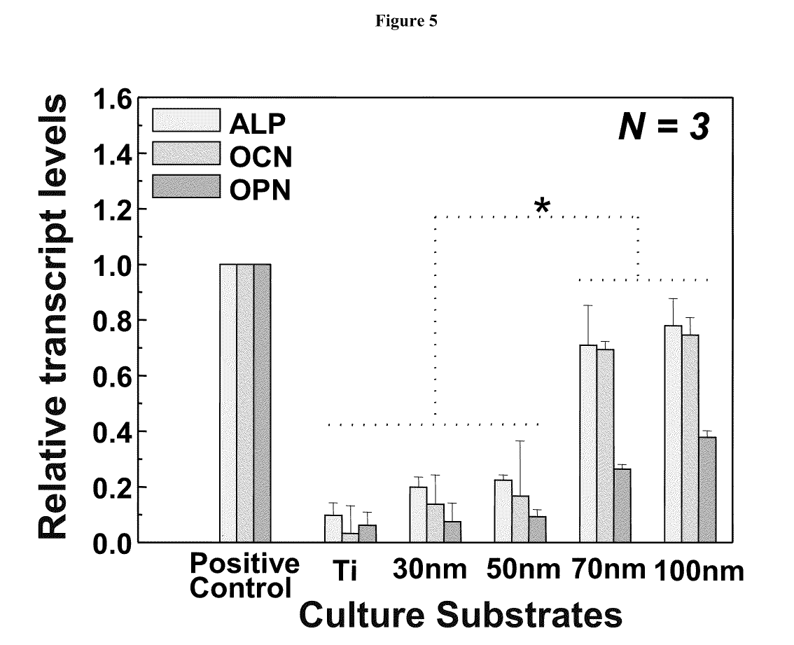

To further support the immunofluorescent staining results described above, osteoblast gene expression (osteocalcin: OCN, osteopontin: OPN, and alkaline phosphatase: ALP) was also studied by quantitative real-time PCR analysis after 14 days of incubation, with the analysis data shown in FIG. 5 (see additional figure discussion, below). hMSCs on 70 and 100 nm TiO₂ nanotubes of the invention demonstrated various levels of osteogenic up-regulation and showed significantly higher level of expression than those on other substrata (p<0.01). 100 nm nanotubes of the invention displayed the highest up-regulation of all selected osteoblastic genes among the experimental groups at just 14 days of incubation and were closest to chemically-induced osteoblast gene expression (positive control). Together, immunofluorescence and real-time PCR results seemed to confirm that 100 nm nanotubes have the potential as a guided differentiation tool for directing hMSCs into osteoblast-like cells in the absence of osteogenesis-inducing media. In contrast, the 30 nm nanotube sample exhibited a significantly lower degree of almost negligible osteoblast gene expression (considering the magnitude of error bars), as indicated in FIG. 5.

Among the various sizes of TiO₂ nanotubes, the smaller dimension nanotubes of 30 nm diameter (dia), and to some extent for the 50 nm TiO₂ nanotubes of the invention showed a much higher population of protein aggregates on the top surface when compared to nanotubes of the invention with 70 nm or 100 nm diameter. The 30 nm diameter nanotubes in particular elicit complete substrate surface coverage with the protein aggregates. The reason for the much reduced protein aggregate adsorption observed on the top of larger diameter nanotubes of the invention of 70 and 100 nm dia. is most likely due to the fact that approximately 30 nm size protein aggregates are just too small to anchor on 70 or 100 nm diameter nanopores with much larger open pore spaces, as the protein aggregates initially attach only onto the available surfaces which are the top portion of the nanotube walls. We have found a threshold point of nanotube size (approximately ≦50 nm) where the adhesion of proteins (approximately 30 nm size regime) initially determines the degree of cell adhesion, a critical observation in this invention.

While the invention is not limited by any particular mechanism of action, the phenomenon of initial protein adsorption is useful in understanding why hMSCs were more elongated on the 100 nm nanotube surfaces of the invention than hMSCs on the smaller sized nanotube surfaces. Extracellular matrix (ECM) proteins are required for a cell to adhere to the surface and to be able to spread out. Within an initial incubation time (say, less than 6 hrs), the proteins available in the culture media from the serum adsorb to the surface and act as the preliminary ECM, which can help cell adhesion considerably at the earliest stages of culture before cells begin secreting their own ECM. hMSCs cultured on less than 50 nm TiO₂ nanotubes can more easily attach to the surface due to the high population of ECM proteins deposited across the entire surface as indicated in FIG. 3. However, hMSCs cultured on 100 nm TiO₂ nanotubes probably would have to struggle to search for areas where more protein aggregates have been deposited in order to establish initial contract because there is much more inter-protein aggregate spacing due to the large pore sizes in the nanostructure. Eventually, the hMSCs have to extend their filopodia extensions excessively and move to find proteins, thus becoming more mobile and forming more extraordinarily elongated shapes.

It is notable that the hMSCs adhered to the smallest dimensioned 30 nm nanotubes exhibit a higher protein population initially on the surface. However in comparison, the larger diameter nanotubes of the invention (e.g., 70 and 100 nm diameter) induced a dramatic cell elongation and also increased osteogenic expressions (see e.g., FIGS. 4 and 5) with a lower amount of adhered cells.

In alternative embodiments, the compositions of the invention are used to induce differentiation in the absence of additional biochemical inducing agents; in this embodiment, this observed attainment of differentiation in the absence of additional biochemical inducing agents can be critical to the success of this invention's embodiment comprising nano-topography-induced differentiation, as shown in results described herein.

In alternative embodiments, the compositions of the invention comprise a larger-diameter nanotube approach; which in some embodiments relies on controlling stem cell fate by using only the nano-topography of the product of manufacture. In some embodiments, this is much more dependable and possibly safer than depending on differentiation-inducing agents or chemical. Thus, when practicing these embodiments of the invention the practitioner can expect a similar and more reproducible in vivo response because these embodiments (using only the nano-topography) do not have the in vivo variability and side effects associated with toxic chemical additions.

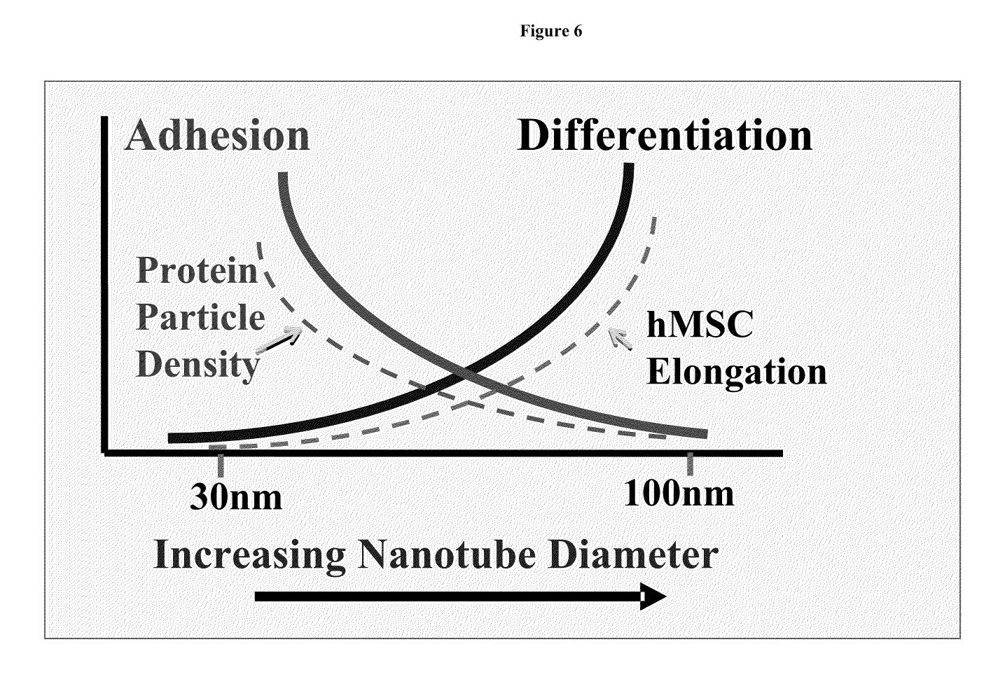

While the invention is not limited by any particular mechanism of action, trends of hMSCs adhesion, elongation, and differentiation behavior disclosed herein as a function of nanotube dimension seem to reveal a specific mechanism which determines the stem cell fate solely on the geometric cues of the surface. While the invention is not limited by any particular mechanism of action, the mechanism follows that on small diameter nanotubes, increased cell adhesion and growth with minimal differentiation seems to be prevalent because this is in part due to the protein aggregate adhesion configurations induced by the small nanotubes. On the larger diameter nanotubes (as used by embodiments of this invention, as described herein), hMSC cells are forced to elongate and stretch to search for protein aggregates, and as a result, are forced/guided to differentiate specifically into osteoblast cells, as illustrated in FIG. 6 (see additional figure discussion, below). When stem cells are stressed due to the high elastic modulus for example, stiff substrates, differentiation into osteoblast cells is enhanced.

Alternative embodiments of this invention control TiO₂ nanotube diameter to optimally guide, induce or direct a mesenchymal stem cell's osteogenic differentiation, which can be desirably induced even in the absence of osteogenic chemical or growth factor media for the exemplary about 70 to 100 nm diameter TiO₂ nanotubes of the invention. The effectiveness of these embodiments of the invention (and data described herein) demonstrate that the cell adhesion and growth (without differentiation) vs. guided differentiation is strongly correlated with the size of nanotubes in a relatively small window of nanotube diameter ranges. Data described herein demonstrates that a guided osteogenic differentiation of hMSCs can be controllably manipulated by selective sizing of the nanotube dimensions. Because the TiO₂ nanotubes are excellent osseo-integration biomaterials, as evidenced by in vitro and in vivo animal data, embodiments of the invention using TiO₂ nanotubes can be used to initiate a strong new bone integration on a nanotube surface with reduced soft tissue trapping. Thus, for these embodiments of the invention, the nanotube surface can perform dual therapeutic functions of specifically guided differentiation and strong osseo-integration for new bone formation.

### Materials and Methods

TiO₂ nanotube Fabrication: The protocol for preparation of TiO₂ nanotubes by anodization process for cell culture is similar to that described in WO 2006/116752 A2; and WO 2008/066965 A2, e.g., describing fabrication of a TiO₂. For example:

The nanotubes were formed on a Ti sheet, (Alfa-Aesar, 0.25 mm thick, 99.5%) by using a mixture of 0.5 wt % hydrofluoric acid (EM Science, 48%) and acetic acid (Fisher, 98%, Volumetric Ratio=7:1) at 5, 10, 15 and 20 V for 30 minutes to obtain different diameter nanotubes. A platinum electrode (Alfa-Aesar, 99.9%) served as the cathode. The specimen was rinsed by D.I. water, dried at 80° C., and heat treated at 500° C. for 2 hours to transform the as-anodized amorphous TiO₂ nanotubes into crystalline phase. The specimens (1.27×1.27 cm² area) used for all experiments were sterilized by autoclaving before use. An identical sized flat Ti sample was used as a control after being chemically cleaned by acetone and isopropanol, dried, and autoclaved.

Cell Culture Human mesenchymal stem cells (hMSCs) were obtained from Lonza Corporation. The cell growth media were composed of α-MEM (Invitrogen), 10% Fetal Calf Serum (FCS) (Invitrogen), 100 units/ml penicillin and 100 ug/ml streptomycin (Invitrogen). For preparing positive control in this research, osteogenic inducing media were also prepared by adding 10 nM dexamethasone (Sigma), 150 μg/mL-Ascorbic acid (Sigma), and 10 mM beta-glycerophosphate (Calbiochem) to cell growth media (see e.g., references 33, 34). The cells were cultured in a 5% CO₂ incubator at 37° C. All experiments of hMSCs were conducted with cultures at passage 4.

Scanning electron microscopy (SEM) for substrate and cell morphological examination: Initially cells were plated on the substrates at a density of 1×10⁴ cells/ml. After 2 hours of culture, the cells on the substrates were washed with 1×PBS and fixed with 2.5% glutaraldehyde in 0.1×PBS for 1 hour. After fixation, they were washed three times with 1×PBS for 10 minutes each wash. Then the cells were dehydrated in a graded series of ethanol (50, 70, 90, and 100%) for 30 minutes each and left in 100% ethanol until they were dried by supercritical point CO₂. Next, the dried samples were sputter-coated with very thin gold for SEM (scanning electron microscopy) examination. The morphology of the TiO₂ nanotubes as well as that of the adhered cells were observed using SEM (XL30, FEI Corporation, Hillsboro, Oreg.).

Immunofluorescence of Actin and Osteopontin/Osteocalcin: After 3 weeks of culture, the cells on the Ti and TiO₂ nanotube substrates were fixed in 4% paraformaldehyde in 1×PBS for 15 minutes at room temperature. Once fixed, the cells were washed twice with 1× wash buffer (1×PBS containing 0.05% Tween-20). To permeabilize the cells 0.1% Triton X-100 in 1×PBS solution was added for 10 minutes. The cells were washed twice with wash buffer. Then the samples were incubated for 1 hour at room temperature in 1% BSA/1×PBS followed by the addition of anti-osteopontin (OPN) antibody (1:100, AKm2A1, Santa Cruz Biotech. Inc.)/anti-osteocalcin (OCN) antibody (1:100, OC4-30, QED Bioscience Inc.), and incubated for overnight at 4° C. After incubation, cells were washed three times for 5 minutes each wash with 1× wash buffer. Goat anti-mouse IgG-FITC (1:100, Santa Cruz Biotech., Inc.) and TRITC-conjugated phalloidin (1:40, Invitrogen) in 1×PBS was added for double staining and the cells were incubated again for 1 hour at room temperature. The cells were washed three times with 1× wash buffer for 5 minutes each wash. Then, the samples were stained by DAPI (1:1000, Chemicon) for nucleus staining. The samples were then inverted onto cover-slips mounted, visualized and photographed by an epifluorescence microscope (DM IRB, Leica Microsystems Inc., Bannockburn, Ill.).

Real-time PCR: After 2 weeks of culture, total RNA of the cells on the Ti and TiO₂ nanotube substrates were extracted with Trizol (Sigma), and reverse-transcribed into cDNA by qSCRIPT™ cDNA Synthesis Kit (Quanta BioSciences) Real-time PCR was performed by TAQMAN® Gene Expression Assays (Applied Biosystems), and the information of TAQMAN® PCR primer is as follows; GAPDH (Hs99999905 ml, Amplicon length: 122), ALP (Hs01029141_g1, Amplicon length: 71), OCN (Hs00609452_g1, Amplicon length: 74) and OPN (Hs00960942 ml, Amplicon length: 63). Real-time PCR were carried out using TAQMAN® FAST UNIVERSAL PCR MASTER MIX® and 7900 HT FAST REAL-TIME PCR SYSTEM® (Applied Biosystems, Foster City, Calif.). cDNA samples (1 μl for total volume of 20 μl) were analyzed for gene of interest and for house-keeping gene GAPDH. The comparison test of cycle-threshold point was used to quantify the gene expression level of each sample. In this study, all levels of expression were normalized by the level of expression of positive control (hMSCs cultured with osteogenic inducing media).

Statistical analysis: In terms of cell count, cell elongation, and real-time PCR assay, all data were expressed as mean±standard error, and analyzed statistically by paired Student t-test method. Significant difference was determined at p-values at least less than 0.01.

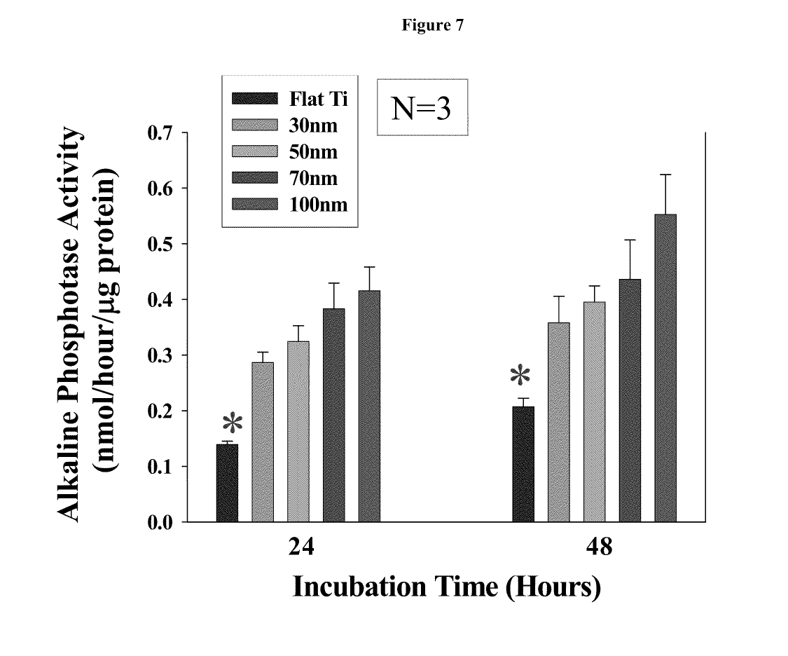

In alternative embodiments, the invention provides compositions and methods that enhance or “guide” the osteogenic functionality of primary osteoblast cells by control of TiO₂ nanotube diameter to larger regime of between about 70 to 100 nm diameter, as graphically illustrated in FIG. 7 (see additional figure discussion, below). The alkaline phosphatase activity (ALP) of osteoblasts, an in vitro marker of bone formation capability, increased proportionally with increasing size of the TiO₂ nanotube diameters. The 100 nm TiO₂ nanotubes induced much increased elongation and the highest ALP functionality. The results of ALP demonstrate that 100 nm TiO₂ nanotubes having the most increased biochemical activity of osteoblast cells can promote the greatest integration of material into surrounding bone than compared with smaller diameter TiO₂ nanotubes.

In alternative embodiments the matrix material that constitutes the nanotube arrays or nanowire, nano-line or nano-groove arrays can be made of a metal or a metal alloy comprising an elemental metal such as Ti, Zr, Hf, Nb, Ta, Mo and/or W metal material, or a Ti, Zr, Hf, Nb, Ta, Mo and/or W alloy that optionally contains other elements, an example being the well known Ti-6% Al-4% V implant alloy. The matrix can also be made of Ti, Zr, Hf, Nb, Ta, Mo and/or W oxide or nitride that optionally contains other elements.

In one embodiment, these metallic or oxide materials are alternatively coated on the surface of other matrix materials having the desired nanotube arrays or nanowire, nano-line or nano-groove arrays with desired geometry in order impart similar bone-growth-enhancing characteristics or enhanced osteogenic differentiation behavior.

In one embodiment the nanotubular structures (e.g., including nanotubes, nanowires, nano-lines and/or nano-grooves) can be straight, curved and/or bent, and optionally the nanotubular structures (e.g., including nanotubes, nanowires, nano-lines and/or nano-grooves) are fixed or loosely placed, or a combination thereof, on the biocompatible surface.

In alternative embodiments dimensions of the nanotubular structures (e.g., including nanotubes, nanowires, nano-lines and/or nano-grooves) that are utilized in this invention can be between about 70 to 200 nanometers (nm) in diameter, or between about 60 to 150 nm in diameter, or have a spacing between them of between about 70 to 200 nanometers (nm) in diameter, or between about 60 to 150 nm.

In alternative embodiments dimensions of the nanostructures (e.g., nanotubular structures, nanowires, nano-lines or nano-grooves) used to practice the invention are about 100 nanometers (nm) in diameter, or are between about 80 to 120 nm in diameter, or alternatively the spacing between the nanostructures (e.g., nanotubular structures, nanowires, nano-lines or nano-grooves) can be about 100 nanometers (nm), or about 80 to 120 nm.

In alternative embodiments a desired height of the nanostructures are in the range of approximately 50 to 2000 nanometers, or can be in the range of 100 to about 1000 nanometers. In alternative embodiments a lower limit provides a well defined and biologically effective nanotubes, nanowires, nanolines or nano-grooves, while the upper limit is to reduce the thickness of the bone-growth-enhancing layer on Ti implant surface for the purpose of minimizing mechanical stress accumulations associated with having dissimilar materials and to prevent interface cracking and delamination.

In alternative embodiments nanostructured matrix materials have nanotube arrays or nanowire, nano-line or nano-groove arrays with desired geometry in order impart similar bone-growth-enhancing characteristics or enhanced osteogenic differentiation behavior. In alternative embodiments the invention can also have nanodepot characteristics to controllably release biological agent from the implant surface. The matrix materials providing the nanodepot can be made of metal, oxide, hydroxyapatite, bio-degradable polymer, bio-compatible or bio-inert bone cement, wherein one or more of components selected from a list of the stem cells, osteogenic inducing agent, or the biological agent or therapeutic composition, or growth factor, collagen, nucleic acid, antibiotic, hormone, drug, magnetic particle, metallic particle, ceramic particle, polymer particle or drug delivery particle are stored in a nanotube cavity, or are stored in spacing between adjacent nanotubular structures, nanowires, nano-lines or nano-grooves.

In alternative embodiments stored agents comprise stem cells, osteogenic growth factors or antibiotics, can be released over a desired time span for maximum medical/therapeutic efficiency. The entrance diameter to the nanocavity or nanospacing can be controlled (can be made narrower) for better control of release rate. An external stimulation, such as DC or AC magnetic field (for the nanodepots containing magnetic nanoparticles), electrical field, ultrasonic, infrared photonic stimulations, can optionally be utilized for remote, on-demand release of the stored biological agents.

In alternative embodiments materials, processes and devices of this invention can be utilized in orthopedic applications such as hip implants, spinal repairs, knee implants, dental or periodontal implants, as well as for various tissue engineering medical applications.

### FIGURE SUMMARY

FIG. 1(a)-(d) illustrate SEM micrographs (top view) of exemplary self-aligned TiO₂ nanotubes of the invention with significantly different diameters. The self-assembly layers were generated by anodizing Ti sheets. The images show highly ordered, vertically aligned nanotubes with four different nanotube pore diameters, 30, 50, 70, and 100 nm (FIG. 1(a)-(d), respectively), created by controlling anodizing potentials ranging from 5 to 20 V. FIG. 1(e) illustrates SEM micrographs (oblique view) of exemplary 100 nm diameter TiO₂ nanotubes of the invention, and FIG. 1(f) illustrates cross-sectional transmission electron microscopy (TEM) of the exemplary 100 nm diameter TiO₂ nanotubes of the invention. All scale bars: 200 nm.

FIG. 2 illustrates SEM micrographs of human mesenchymal stem cells (hMSCs) on exemplary flat Ti and 30 nm, 50 nm, 70 nm, 100 nm diameter TiO₂ nanotube surfaces of the invention after 2 hours and 24 hours of culture (Scale bar: 100 μm for all images). Cells are flat, spread-out, and round-shaped on the flat Ti substrate, somewhat flat and rounded on exemplary 30 nm nanotubes, and become progressively elongated as the nanotube diameter is increased to 50 nm dia. and beyond. Extraordinary cell elongation is induced on exemplary nanotubes with diameters of 70 and 100 nm (see red arrows), especially after the 24 hr culture. More mobile morphologies are indicated by the presence of somewhat elongated leading edges of lamellipodia (yellow arrows, or the arrows in the “two hours of incubation” 100 nm view, and the “24 hours of incubation” 70 nm view lower right hand arrow; and “24 hours of incubation” 100 nm view upper arrow), as seen on 70 and 100 nm nanotubular surfaces. The cell shapes suggest that cells are more elongated on the bigger TiO₂ nanotubes.

FIG. 3 illustrates SEM micrographs showing extracellular matrix aggregates on the surfaces of exemplary flat Ti (top left SEM panel) and 30 nm (top middle SEM panel), 50 nm (top right SEM panel), 70 nm (bottom left SEM panel), 100 nm (bottom right SEM panel) diameter TiO₂ nanotubes of the invention after 2 hours of hMSCs culture (scale bar: 200 nm). Note that the presence of protein aggregates is infrequent on Ti, abundant on 30 nm nanotubes, and much less on the larger diameters of 70 and 100 nm nanotubes.

FIG. 4 illustrates immunofluorescent images of osteopontin (OPN) (top middle panel) and osteocalcin (OCN) (bottom middle panel), as well as actin (upper and lower left panels) and DAPI (upper and lower right panels), on exemplary 100 nm diameter TiO₂ nanotubes of the invention after 3 weeks of culture. (Scale bar: 50 μm)

FIG. 5 graphically illustrates data from a quantitative PCR analysis for alkaline phosphatase (ALP), osteocalcin (OCN), and osteopontin (OPN). Plastic cell culture plate with osteogenic inducing media was used as a positive control for osteogenic differentiation. The * indicates significant differences between Ti, the exemplary 30 and 50 nm nanotubes vs. the exemplary 70 and 100 nm nanotubes of the invention for ALP, OCN, and OPN gene expressions. (p<0.01).

FIG. 6 is a schematic illustration of the overall trends of nano cue effects of products of manufacture of the invention on hMSC fate and morphology after 24 hr culture. The change in hMSC cell adhesion and growth without differentiation (solid red line) has the same trend as protein particle density (broken red line), whereas that of differentiation (solid blue line) has the same trend as hMSC elongation (broken blue line).

FIG. 7 graphically illustrates alkaline phosphatase activity (as nmol/hour/μg protein) of primary bone cells (osteoblast cells) cultured on flat Ti, and various sized TiO₂ nanotubes of the invention (at 30, 50, 70 and 100 nm), at 24 hour (h) (left) and 48 h (right) of incubation. The bar graphs show the average±standard error bars.

It should be understood that the invention can be practiced with modification and alteration within the spirit and scope of the appended claims. The description is not intended to be exhaustive or to limit the invention to the precise form disclosed. It should be understood that the invention can be practiced with modification and alteration and that the invention be limited only by the claims and the equivalents thereof.

What is claimed:

1. A product of manufacture comprising a biocompatible surface,
wherein at least a portion of (or at least part of) (e.g., at least about 0.5%, 1%, 2%, 3%, 4%, 5%, 6%, 7%, 8%, 9% or 10% or more), or all of (or the entirety, or 100% of), the surface area of the biocompatible surface comprises or is covered or coated by a structure or structures comprising:
(a) (i) a plurality of nanotubular structures that are between about 70 to 200 nanometers (nm) in diameter, or between about 60 to 150 nm in diameter
(ii) a plurality of nanowires, nano-lines or nano-grooves having a spacing of between about 70 to 200 nanometers (nm), or between about 60 to 150 nm, or
(iii) a combination (in any proportion or combination) of the nanotubular structures of (i) and the nanowires, nano-lines or nano-grooves of (ii);
(b) the product of manufacture of (a), wherein a portion of, or all of, the nanotubular structures comprise nanotubes;
(c) the product of manufacture of (a) or (b), wherein the nanotubular structures, nanowires, nano-lines or nano-grooves comprise a metal or a metal alloy, and/or a stainless steel or a ceramic, and/or the biocompatible surface (e.g., as a coating) comprises a metal or a metal alloy, and/or a stainless steel or a ceramic, and/or a polymer;
(d) the product of manufacture of (c), wherein the metal or a metal alloy comprises Ti, Zr, Hf, Nb, Ta, Mo and/or W metal material, a Ti, Zr, Hf, Nb, Ta, Mo and/or W alloy, a Ti, Zr, Hf, Nb, Ta, Mo and/or W single or alloyed oxide,
wherein the metal, metal alloy, metal material, single or alloyed oxide comprise at least part of (e.g., at least about 1%) or the entire materials content of a nanostructure, or are manufactured as all (as completely, or 100%) or part of (e.g., at least about 1%) a surface coating with between about 2 to 20 nm, or between about 1 to 40 nm, or between about 0.5 to 60 nm, thickness on the nanostructures (e.g., nanotubes, nanowires, nano-lines or nano-grooves);
(e) the product of manufacture of any of (a) to (d), wherein the nanotubular structures, nanowires, nano-lines or nano-grooves are straight, curved and/or bent, and optionally the nanotubular structures, nanowires, nano-lines or nano-grooves are fixed or loosely placed, or a combination thereof, on the biocompatible surface (which may be a coating);
(f) the product of manufacture of any of (a) to (e), wherein at least a portion of, or all of, the nanotubular structures, nanowires, nano-lines and/or nano-grooves are arranged as an array, and optionally the nanotubular structures, nanowires, nano-lines and/or nano-grooves are arranged as three-dimensional network scaffolds;
(g) the product of manufacture of any of (a) to (f), wherein at least a portion of (or at least part of) (e.g., at least about 0.5%, 1%, 2%, 3%, 4%, 5%, 6%, 7%, 8%, 9% or 10% or more), or all of (or the entirety, or 100% of), the nanotubular structures, nanowires, nano-lines or nano-grooves are in contact with or are contained within or upon (e.g., in contact with the surface of) or within the immediate vicinity of a cell, wherein optionally the cell is suitable for implantation and/or regeneration of a bone and/or a joint tissue in a subject, and optionally the cell is an osteoblast, a stem cell or a mesenchymal stem cell (MSC);
(h) the product of manufacture of any of (a) to (g), wherein at least a portion of (or at least part of) (e.g., at least about 0.5%, 1%, 2%, 3%, 4%, 5%, 6%, 7%, 8%, 9% or 10% or more), or all of (or the entirety, or 100% of), the nanotubular structures, nanowires, nano-lines or nano-grooves comprise or further comprise (e.g., have contained therein, or are associated with, or are covalently or non-covalently joined to or associated with) one or more agents or compositions, or a biologically active agent, or an osteogenic inducing agent, or a therapeutic drug, a growth factor, a protein, an enzyme, a hormone, a nucleic acid, an RNA, a DNA, a gene, a vector, a phage, an antibiotic, an antibody, a small molecule, a radioisotope, a magnetic nanoparticle and/or a particle;
(i) the product of manufacture of any of (a) to (h), wherein the nanotubular structures, nanowires, nano-lines or nano-grooves are between about 70 to 200 nanometers (nm) in diameter or width, or between about 60 to 150 nm in diameter or width, or have a spacing between the nanowires, nano-lines or nano-grooves of between about 70 to 200 nanometers (nm), or between about 60 to 150 nm; and optionally the nanotubular structures, nanowires, nano-lines or nano-grooves are in contact with or are contained within or upon (e.g., in contact with the surface of) or within the immediate vicinity of a cell, or a stem cell, or a mesenchymal stem cell (MSC) or a human mesenchymal stem cell (hMSC), or an embryonic stem cell, or
(j) the product of manufacture of any of (a) to (h), wherein the nanotubular structures, nanowires, nano-lines or nano-grooves are about 90, 95, 100, 105 or 110 or more nanometers (nm) in diameter or width, or are between about 80 to 120 nm in diameter or width, or have a spacing between the nanowires, nano-lines or nano-grooves of about 100 nanometers (nm), or have a spacing between them of about 80 to 120 nm; and optionally the nanotubular structures, nanowires, nano-lines or nano-grooves are in contact with or are contained within or upon (e.g., in contact with the surface of) or within the immediate vicinity of a cell, or a stem cell, or a mesenchymal stem cell (MSC) or a human mesenchymal stem cell (hMSC), or an embryonic stem cell, or a “regular” or fully differentiated or partially differentiated osteoblast cell.

2. The product of manufacture of claim 1:
(a) wherein at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or all, of the biocompatible surface is covered by a plurality of nanotubular structures, nanowires, nano-lines or nano-grooves;
(b) wherein the biocompatible surface and/or the nanotubular structures, nanowires, nano-lines or nano-grooves comprise: (i) a matrix material; or (ii) a Ti, Zr, Hf, Nb, Ta, Mo and/or W metal; or an oxide of a Ti, Zr, Hf, Nb, Ta, Mo and/or W metal; or (iii) an alloy or alloyed oxide of a Ti, Zr, Hf, Nb, Ta, Mo and/or W metal; or a Si, a Si oxide, an Al, an Al oxide, a carbon, a diamond, a noble metal, an Au, an Ag, a Pt and/or an Al, Au, an Ag, a Pt alloy, a polymer or a plastic material, a composite metal, a ceramic, a polymer and/or a combination thereof, and optionally having a film coating of between about 1 to 40 nm thickness, or between about 2 to 20 nm thickness, and optionally having a film coating comprise (in whole or in part) a Ti, Zr, Hf, Nb, Ta, Mo and/or W metal; or an oxide of a Ti, Zr, Hf, Nb, Ta, Mo and/or W metal; (iii) an alloy or an alloyed oxide of a Ti, Zr, Hf, Nb, Ta, Mo and/or W metal;
(c) further comprising a bone cell, a liver cell, a kidney cell, a blood vessel cell, a skin cells, a periodontal cell or a periodontal tissue cell, a stem cell, an organ cell, or wherein the cell is a bone cell, a liver cell, a kidney cell, a blood vessel cell, a skin cells, an organ cell; or, further comprising a plurality of cells, wherein the cells comprise bone cells, liver cells, liver parenchymal cells, endothelial cells, adipocytes, fibroblastic cells, Kupffer cells, kidney cells, blood vessel cells, skin cells, periodontal cells, odontoblasts, dentinoblasts, cementoblasts, enameloblasts, odontogenic ectomesenchymal tissue, osteoblasts, osteoclasts, fibroblasts, and other cells and tissues involved in odontogenesis or bone formation and/or stem cells, and other human or animal organ cells, or the cells are embryonic or adult stem cells, or a combination thereof;
(d) the product of manufacture of (c), wherein the cell is a human or an animal cell, or the product of manufacture further comprises or contains a human or an animal cell;
(e) further comprising a hydroxyapatite, a bio-degradable polymer, or a bio-compatible or bio-inert bone cement; or further comprising a biological agent or a therapeutic composition, or an osteogenic inducing agent, or a growth factor, a collagen, a nucleic acid, an antibiotic, a hormone, a drug, a magnetic particle, a metallic particle, a ceramic particle, a polymer particle and/or a drug delivery particle;
(f) wherein the nanotubular structures or nanotubes are made by anodization or by patterned chemical etching or a combination thereof;
(g) the product of manufacture of any of (a) to (f), further comprising a nanodepot comprising a metallic or oxide material, hydroxyapatite, a bio-degradable polymer, or a bio-compatible or bio-inert bone cement; or further comprising an agent or a composition, or an osteogenic inducing agent, or a biological agent or a therapeutic composition, or a growth factor, a collagen, a nucleic acid, an antibiotic, a hormone, a drug, a magnetic particle, a metallic particle, a ceramic particle, a polymer particle and/or a drug delivery particle; or
(h) the product of manufacture of any of (a) to (g), wherein the matrix material providing the nanodepot is made of (e.g., comprises entirely or partly of) a metal, a metal oxide, an oxide, a hydroxyapatite, a bio-degradable polymer, a bio-compatible or bio-inert bone cement, wherein one or more compositions, components or agents or cells, e.g., stem cells, osteogenic inducing agents, or a biological agent or therapeutic composition, or growth factor, collagen, nucleic acid, antibiotic, hormone, drug, magnetic particle, metallic particle, ceramic particle, polymer particle and/or a drug delivery particle, are stored or contained in a nanotubular structure or a nanotube cavity, or are stored in or contained in spacing between adjacent nanotubular structures, nanowires, nano-lines or nano-grooves.

3. A composition delivery device comprising (a) a product of manufacture of claim 1, or (b) the product of manufacture of (a), wherein the composition comprises a hydroxyapatite, a bio-degradable polymer, or a bio-compatible or bio-inert bone cement; or further comprises a cell, a stem cell, an osteogenic inducing agent, or a biological agent or a therapeutic composition, or a growth factor, a collagen, a nucleic acid, an antibiotic, a hormone, a drug, a magnetic particle, a metallic particle, a ceramic particle, a polymer particle and/or a drug delivery particle.

4. A medical implant, an orthopedic (orthopedic) implant or a dental implant, or an artificial tissue or organ, comprising: (a) a product of manufacture of claim 1; or (b) the product of manufacture of (a), wherein medical implant, orthopedic (orthopedic) implant or dental implant, or artificial tissue or organ, comprises or has contained within or is coated with, or is associated with, a plurality of cells, and optionally the cells comprise bone cells, liver cells, liver parenchymal cells, endothelial cells, adipocytes, fibroblastic cells, Kupffer cells, kidney cells, blood vessel cells, skin cells, periodontal cells, odontoblasts, dentinoblasts, cementoblasts, enameloblasts, odontogenic ectomesenchymal tissue, osteoblasts, osteoclasts, fibroblasts, and other cells and tissues involved in odontogenesis or bone formation and/or stem cells, and other human or animal organ cells, or the cells are embryonic or adult stem cells, or a combination thereof.

5. A method for guiding, controlling and/or directing the differentiation of a cell, a mammalian cell, a stem cell, or accelerating the differentiation and directing the differentiation of a cell, a mammalian cell, a stem cell, comprising:
(a) implanting, growing and/or culturing the cell, or mammalian cell, or stem cell, in or on (or on the surface of) a product of manufacture of claim 1;
(b) the method of (a), wherein the method is for guiding, controlling and/or directing the differentiation of the stem cell, or accelerating and directing the differentiation of a cell, or stem cell, or a mesenchymal stem cell (MSC) or a human mesenchymal stem cell (hMSC),
wherein optionally the cell, stem cell, mesenchymal stem cell (MSC) or the human mesenchymal stem cell (hMSC) is provided approximate to (or near or adjacent to) the implant location by injection, by mixing with a composite liquid comprising viscosity-enhancing medium such as a bioinert polymer or a biodegradable polymer in liquid or particle form, by adding to a bone cement, by inserting/storing into nanopores in the nanotubes, and/or by inserting/storing in microcavities or microdepots prepared on a surface of a product of manufacture of the invention, e.g., on an implant surface;
(c) the method of (a) or (b), wherein the nanotubular structures, nanowires, nano-lines or nano-grooves are between about 70 to 200 nanometers (nm) in diameter or width, or between about 60 to 150 nm in diameter or width, or have a spacing between the nanowires, nano-lines or nano-grooves of between about 70 to 200 nanometers (nm), or between about 60 to 150 nm;
(d) the method of any of (a) to (c), wherein the cell, or the stem cell, or the mesenchymal stem cell (MSC), or the human mesenchymal stem cell (hMSC), is directed to differentiate into an osteoblast, or a cell of osteogenic lineage; or
(e) the method of any of any of (a) to (d), wherein the product of manufacture is placed or implanted in vivo, and the mesenchymal stem cell (MSC) or the human mesenchymal stem cell (hMSC) differentiates either before in vivo implantation, after in vivo implantation, or a combination thereof.

6. A method for inducing, enhancing and/or prolonging the bone-forming capacity of a “regular”, or a fully differentiated or a partially differentiated osteoblast cell or cell of osteogenic lineage, comprising:
(a) implanting, growing and/or culturing an osteoblast cell or a cell of osteogenic lineage in a product of manufacture of claim 1;
(b) the method of (a), wherein the nanotubular structures, nanowires, nano-lines or nano-grooves are about 90, 95, 100, 105 or 110 or more nanometers (nm) in diameter or width, or are between about 80 to 120 nm, or 90 to 110 nm or 60 to 140 nm in diameter or width, or have a spacing between the nanowires, nano-lines or nano-grooves of about 90, 95, 100, 105 or 110 or more nanometers (nm), or have a spacing between them of between about 80 to 120 nm, or 90 to 110 nm or 60 to 140 nm;
(c) the method of (a) or (b), wherein the product of manufacture is placed or implanted in vivo;
(d) the method of any of (a) to (c), wherein the metallic or oxide matrix, hydroxyapatite, bio-degradable polymer, bio-compatible or bio-inert bone cement contains or comprises nanostructures, and/or comprises or further comprise an agent or a composition, or a biological agent or a therapeutic composition, or growth factor, collagen, nucleic acid, antibiotic, hormone, drug, magnetic particle, metallic particle, ceramic particle, polymer particle, drug delivery particle, and optionally the agents or compositions, or biological agents or therapeutic compositions, or growth factor, collagen, nucleic acid, antibiotic, hormone, drug, magnetic particle, metallic particle, ceramic particle, polymer particle, drug delivery particle are stored or contained within a nanodepot, or a nanotubular structures or a nanotube cavities, or stored in spacing between adjacent nanowires nanotubular structures, nanowires, nano-lines or nano-grooves, or released by a remote-controlled mechanism or device, and optionally the remote-controlled mechanism or device comprises use of an electric field, a magnetic field, an AC magnetic field, ultrasonic agitation, or infrared light stimulation; or
(e) the method of any of (a) to (d), wherein the guiding, controlling and/or directing of stem cell differentiation and/or inducing, enhancing and/or prolonging of bone formation is improved, accelerated or prolonged by combining with a full-dose or partial use of an osteogenic inducing agent.

7. A method for selectively releasing a therapeutic, an imaging, a drug or a biological agent in a subject, the method comprising
(a) implanting a product of manufacture of claim 1, in a subject, wherein the product of manufacture comprises a therapeutic, an imaging, a drug or a biological agent in a liquid or colloidal composition; and,
(b) contacting the product of manufacture with ultrasonic or magnetic agitation of the liquid or colloidal composition, wherein the biological agent is released from the product of manufacture;
and optionally the magnetic nanoparticle is selected from the group consisting of iron-oxide particles of magnetite (Fe₃O₄) or maghemite (γ-Fe2O3), and optionally the magnetic nanoparticle is about 5 to 50 nm in average diameter.

8. A method for guiding, controlling and/or directing the movement of a cell, a mammalian cell, a stem cell, comprising:
(a) implanting, growing and/or culturing the cell, or mammalian cell, or stem cell, in or on (or on the surface of) or adjacent to, a product of manufacture of claim 1;
(b) the method of (a), wherein the method is for guiding, controlling and/or directing the movement of the stem cell, or accelerating and directing the movement of a cell, or a mesenchymal stem cell (MSC) or a human mesenchymal stem cell (hMSC);
(c) the method of (a) or (b), wherein the nanotubular structures, nanowires, nano-lines or nano-grooves are between about 70 to 200 nanometers (nm) in diameter or width, or between about 60 to 150 nm in diameter or width, or have a spacing between the nanowires, nano-lines or nano-grooves of between about 70 to 200 nanometers (nm), or between about 60 to 150 nm;
(d) the method of any of (a) to (c), wherein the nanotubular structures, nanowires, nano-lines or nano-grooves that are between about 70 to 200 nanometers (nm) in diameter or width, or between about 60 to 150 nm in diameter or width, or have a spacing between the nanowires, nano-lines or nano-grooves of between about 70 to 200 nanometers (nm), or between about 60 to 150 nm, are patterned on the surface of the product of manufacture to direct the movement and/or differentiation of the cell.
